(19) United States
(12) Patent Application Publication
(10) Pub. No.: US 2007/0298136 A1
(43) Pub. Date: Dec. 27, 2007

(54) Cholesterol regulating agent

(76) Inventors: Yoshinori Aoki, Tokyo (JP); Hiroyuki Yoshimura, Tokyo (JP)

Correspondence Address: John L. Cordani, Esq.;Carmody & Torrance, LLP, 50 Leavenworth Street, P.O. Box 1110, Waterbury, CT, 06721-1110, US

(21) Appl. No.: 11/888,938

(22) Filed: Aug. 3, 2007

Related U.S. Application Data
(62) Division of application No. 11/484,182, filed on Jul. 11, 2006.

(30) Foreign Application Priority Data
Jul. 11, 2005 (JP) 2005-202109

(51) Int. Cl. A61K 36/63; A61P 9/14

(52) U.S. Cl. 424/769

(57) ABSTRACT

It is an object of the present invention to provide a HDL cholesterol-raising agent which can selectively raise or maintain HDL cholesterol level, an LDL cholesterol-lowering agent which can selectively lower LDL cholesterol level, a composition which can selectively raise of maintain HDL cholesterol level, a method for raising or maintaining HDL cholesterol level in blood and a method which can raise or maintain HDL cholesterol level and lower LDL cholesterol level in blood. Therefore, provided are a HDL cholesterol-raising agent and LDL cholesterol-lowering agent containing an olive extract as an active ingredient, a composition containing the HDL cholesterol-raising agent, and a method for raising HDL cholesterol level and a method for raising HDL cholesterol level and lowering LDL cholesterol level in which olive extracts are administered to mammals.

### BACKGROUND OF THE INVENTION

1. Field of the Invention

The present invention relates to a cholesterol regulating agent and the cholesterol regulating agent raises HDL cholesterol level and lowers LDL cholesterol level. Specifically, the cholesterol regulating agent is any one of a HDL cholesterol-raising agent and a LDL cholesterol-lowering agent. The present invention also relates to a composition containing the HDL cholesterol-raising agent, and a method for raising HDL cholesterol level in blood and a method for raising HDL cholesterol level and lowering LDL cholesterol level in blood.

2. Description of the Related Art

It is believed that prevention or treatment for onset of circulatory system diseases such as arteriosclerosis, angina pectoris, myocardial infarction, cerebral infarction, etc. can be provided by a therapeutic agent for hyperlipidemia which lowers cholesterol level, a blood lipid and neutral fat, etc.

The low-density lipoprotein (herein after, referred to as “LDL”) cholesterol in blood works to accelerate onset of arteriosclerosis by transporting the hepatic cholesterol in blood.

In contrast, high-density lipoprotein (herein after, referred to as “HDL”) cholesterol in blood works to prevent onset of arteriosclerosis by lowering the cholesterol level in blood.

Therefore, in order to effectively prevent arteriosclerosis, lowering LDL cholesterol level in blood and raising HDL cholesterol level in blood are thought to be effective.

Although expression of the effect is certain for general therapeutic agents for hyperlipidemia, there are problems of side effects or interaction with other medicines, etc. and the development of the therapeutic agent for preventing hyperlipidemia, which contains a food component as a raw material without side effects is strongly desired.

In recent years, olives have received attention as a natural origin substance capable of improving hyperlipidemia. The olives are evergreen trees of olive genus and they are known by their scientific name “Olea europaea Linne” and originate in Mediterranean. The olive oil obtained from the fruit has been used for food through the ages and it is widely known that the oleic acid in the olive oil has LDL cholesterol-lowering effect. In addition to that, it has also been reported that the olive extract has lowering effect on total cholesterol level and neutral fat level and antioxidant effect on lipid or protein, etc. in food or within the body Gapanese Patent Application Laid-Open (JP-A) Nos. 2004-352626, 2002-543103 and 9-78061, International Publication No. WO01/45514, JP-A No. 2001-181632, “Igakutoyakugaku” (Japanese Journal of Medicine and Pharmaceutical Science) Vol. 50, No. 3, 335-339, 2003 and N. Bennani-Kabchi et al. Therapie, 54, 717-723, 1999).

Moreover, it has been reported that unsaponifiable substance, Oleuropein and Oleuropein aglycon, etc. that are extracted from olive plants have catecholamine-inducible effect (JP-A Nos. 2004-269426 and 2005-179353).

An anticholesterol agent containing an extract of olive leaf is mentioned in the invention disclosed in the above literature (JP-A No. 2004-352626) and it is stated that water extract of olive leaf has a function to lower total cholesterol level, LDL cholesterol level and VLDL cholesterol level (N. Bennani-Kabchi et al. Therapie, 54, 717-723, 1999).

Although it is stated in these literatures that the extract of olive leaf lowers total cholesterol level or lowers total cholesterol level, LDL cholesterol level and VLDL cholesterol level, it has not yet been reported that the extract of olive leaf selectively raises HDL cholesterol level or selectively lowers LDL cholesterol level.

### SUMMARY OF THE INVENTION

The present invention is intended to settle above existing issues and to achieve the following objects. It is an object of the present invention to provide [1] an HDL cholesterol-raising agent which can selectively raise or maintain HDL cholesterol level, [2] a LDL cholesterol-lowering agent which can selectively lower LDL cholesterol level, [3] a composition containing the HDL cholesterol-raising agent which raises or maintain HDL cholesterol level, [4] a method for raising HDL cholesterol level in blood which selectively raises or maintain HDL cholesterol level in blood of mammals and [5] a method for raising HDL cholesterol level and lowering LDL cholesterol level which selectively raises or maintain HDL cholesterol level and further selectively lowers LDL cholesterol level in blood of mammals.

As a result of dedicated investigation conducted on the above issues, the present inventors have obtained the following knowledge. It has been found that by using an olive extract as an active ingredient, it is possible to obtain an HDL cholesterol-raising agent which can selectively raise or maintain HDL cholesterol level and a LDL cholesterol-lowering agent which can lower LDL cholesterol level. It has also been found that it is possible to obtain a composition which can raise or maintain HDL cholesterol level by containing the HDL cholesterol-raising agent, a method for raising HDL cholesterol level in blood which can raise or maintain HDL cholesterol level in blood by administering an olive extract to mammals, and a method for raising HDL cholesterol level and lowering LDL cholesterol level in blood which raises or maintains HDL cholesterol level and lowers LDL cholesterol level in blood.

The present invention is based on the knowledge of the present inventors and the measures to settle above issues are as follows.

<1> A HDL cholesterol-raising agent comprising an olive extract wherein the olive extract is an active ingredient of the HDL cholesterol-raising agent.

<2> A LDL cholesterol-lowering agent comprising an olive extract wherein the olive extract is an active ingredient of the LDL cholesterol-lowering agent.

<3> A composition comprising a HDL cholesterol-raising agent wherein the HDL cholesterol-raising agent comprises an olive extract as an active ingredient.

<4> A method for raising HDL cholesterol level in blood comprising administration of an olive extract to mammals.

<5> A method for raising HDL cholesterol level and lowering LDL cholesterol level in blood comprising administration of an olive extract to mammals.

### BRIEF DESCRIPTION OF THE DRAWINGS

FIG. 1 is a diagram showing the averages of LDL cholesterol level, total cholesterol level and HDL cholesterol level before and after administration of the HDL cholesterol-raising agent of the present invention.

FIG. 2 is a diagram showing the averages of triglyceride level before and after administration of the HDL cholesterol-raising agent of the present invention.

### DESCRIPTON OF THE PREFERRED EMBODIMENTS

Examples of the ingredients contained in the olive extract, which are active ingredients of the HDL cholesterol-raising agent of the present invention include Oleuropein, Oleuroside, Ligstroside, Oleoside-7, 11-dimethylester, Oleanolic acid, Maslinic acid, Hydroxytyrosol, Chlorogenic acid, Verbascoside, Luteolin, Luteolin-7-glucoside, Rutin, Apigenin-7-glucoside, Apigenin-7-rutinoside, Apigenin, Quercetin, Quercitrin, Luteorin-4′-glucoside, Luteorin-7-rutinoside, and Demethyloleuropein. It is preferable even among these for Oleuropein to be mainly included in the extract.

The content of the Oleuropein relative to the whole amount of the olive extract is preferably at least 25% by mass and more preferably at least 35% by mass. The content of the Oleuropein is adjusted according to the method for manufacturing olive extract and additional amount of diluents contained in the HDL cholesterol-raising agent of the present invention and higher content is better.

The olive extract contained in the HDL cholesterol-raising agent of the present invention can be obtained by extraction methods generally used for plant extraction using the extraction material. The extraction methods are not particularly limited and known methods can be used. Examples include a method in which raw or dry olives are used and solvent extraction is performed directly or after pulverization by means of a pulverizer.

Examples of the method for manufacturing extract include the methods for manufacturing disclosed in JP-A Nos. 2002-128678 and 2003-335693. The methods for manufacturing disclosed in JP-A Nos. 2002-128678 and 2003-335693 may be used simultaneously as the extraction method.

The HDL cholesterol-raising agent of the present invention contains the olive extract as an active ingredient. The HDL cholesterol-raising agent has HDL cholesterol-raising effect because it contains the olive extract as an active ingredient.

The olive extract, which is used as an active ingredient of the HDL cholesterol-raising agent of the present invention, preferably contains the Oleuropein.

The HDL cholesterol-raising effect acts to raise the HDL cholesterol level in blood after administration of the HDL cholesterol-raising agent higher than the HDL cholesterol level in blood before administration of the HDL cholesterol-raising agent, or to maintain the HDL cholesterol level in blood after administration of the HDL cholesterol-raising agent at the HDL cholesterol level in blood before administration of the HDL cholesterol-raising agent.

Moreover, it is preferable for the HDL cholesterol-raising agent of the present invention to additionally have LDL cholesterol-lowering effect. The LDL cholesterol-lowering effect acts to lower the LDL cholesterol level in blood after administration of the HDL cholesterol-raising agent lower than the LDL cholesterol level in blood before administration of the HDL cholesterol-raising agent.

The LDL cholesterol-lowering agent of the present invention contains the olive extract as an active ingredient. The LDL cholesterol-lowering agent has LDL cholesterol-lowering effect because it contains the olive extract as an active ingredient. The LDL cholesterol-lowering effect can lower the LDL cholesterol level after administration of the LDL cholesterol-lowering agent lower than the LDL cholesterol level before administration of the LDL cholesterol-lowering agent.

The intake per day of the HDL cholesterol-raising agent of the present invention is preferably the amount at which 100 mg to 1,000 mg of olive leaf extract can be ingested and more preferably the amount at which 150 mg to 600 mg of olive leaf extract can be ingested. Or, it is preferably the amount at which 30 mg to 500 mg of Oleuropein can be lo ingested and more preferably the amount at which 50 mg to 250 mg of Oleuropein can be ingested.

The route of administration of the HDL cholesterol-raising agent of the present invention is not particularly limited and examples include oral, intravenous, intraarterial, transmucosal, transdermal, transvaginal and abdominal routes, nasal drops and eye drops. Among them, oral administration is preferable.

The HDL cholesterol-raising agent of the present invention can be favorably applied for compositions, medicaments, foods or drinks.

(Composition)

The composition of the present invention contains the HDL cholesterol-raising agent. Because the composition contains the HDL cholesterol-raising agent, it has HDL cholesterol-raising effect. The HDL cholesterol-raising effect acts to raise the HDL cholesterol level in blood after ingestion of the composition higher than the HDL cholesterol level in blood before ingestion of the composition or to maintain the HDL cholesterol level in blood after ingestion of the composition at the HDL cholesterol level in blood before ingestion of the composition.

Moreover, it is preferable for the composition to additionally have LDL cholesterol-lowering effect. The LDL cholesterol-lowering effect acts to lower the LDL cholesterol level in blood after ingestion of the composition lower than the LDL cholesterol level in blood before ingestion of the composition.

The composition of the present invention includes that one which contains the LDL cholesterol-lowering agent. Because the composition contains the LDL cholesterol-lowering agent, it has LDL cholesterol-lowering effect. The LDL cholesterol-lowering effect acts to lower the LDL cholesterol level in blood after ingestion of the composition lower than the LDL cholesterol level in blood before ingestion of the composition.

The composition is not particularly limited and may be selected accordingly and examples include medicaments, medicated cosmetics, foods, drinks and health foods.

The composition may be formed by using known diluents generally used for manufacture of medicaments, foods or drinks.

The formulation of the medicaments is not particularly limited and it may be known formulation such as tablet, pill, powder, micro powder, granulate, syrup, liquid, suspension, emulsion and capsule.

The foods or drinks are not particularly limited and may be selected accordingly. Examples include drinks, frozen desserts, noodles, confectionery, marine products, processed marine and animal products, dairy products, oil and fat and processed oil and fat products, seasoning agents, retort pouch foods, and health and nutritive foods.

In the method for raising HDL cholesterol level in blood of the present invention, the olive extract is administered to mammals. By the method for raising HDL cholesterol level in blood, the HDL cholesterol level in blood of mammals after administration of the olive extract can be raised higher than the HDL cholesterol level in blood of mammals before administration of the olive extract or the HDL cholesterol level in blood of mammals after administration of the olive extract can be maintained at the HDL cholesterol level in blood of mammals before administration of the olive extract.

In the method for raising HDL cholesterol level and lowering LDL cholesterol level in blood of the present invention, the olive extract is administered to mammals. By the method for raising HDL cholesterol level and lowering LDL cholesterol level in blood, the RDL cholesterol level in blood of mammals after administration of the olive extract can be raised higher than the HDL cholesterol level in blood of mammals before administration of the olive extract or the HDL cholesterol level in blood of mammals after administration of the olive extract can be maintained at the HDL cholesterol level in blood of mammals before administration of the olive extract. Furthermore, the LDL cholesterol level in blood of mammals after administration of the olive extract can be lowered lower than the LDL cholesterol level in blood of mammals before administration of the olive extract.

The olive extract preferably contains Oleuropein.

### EXAMPLES

### Example 1

Production of Olive Extract

The olive leaves were dried by a method of Example 3 disclosed in JP-A No. 2003-335693 (by hydrothermal temperature of 80° C.). The extraction of olive leaves was performed according to the method of Example 4 disclosed in JP-A No. 2002-128678 to obtain an olive extract. The content of Oleuropein in the obtained olive extract was 36.2% by mass. The content of Oleuropein was measured by HPLC.

Production of Tablet

By using an olive extract obtained by the production of olive extract as the HDL cholesterol-raising agent of the present invention and using a mixing kneader (“Super Mixer” by Kawata Mfg. Co., Ltd.), 140 g of olive extract obtained in Example 1, 500 g of microcrystalline cellulose (“Avicel™FD-101” by Asahi Kasei Chemicals Corp.), 330 g of indigestible dextrin (“Fibersol-2” by Matsutani Chemical Industry Co., Ltd.) and 30 g of sucrose esters of fatty acid (“DKesterF20W” by Dai-ichi Kogyo Seiyaku Co., Ltd.) were mixed and made into tablets by means of a rotary tableting machine (“AP-15” by Hata Iron Works Co., Ltd.) with a compression pressure of 900 kg and 3,200 tablets were obtained with a weight of 300 mg each. The obtained tablets were coated with shellac and the tablets containing the HDL cholesterol-raising agent of the present invention were obtained.

Evaluation Method

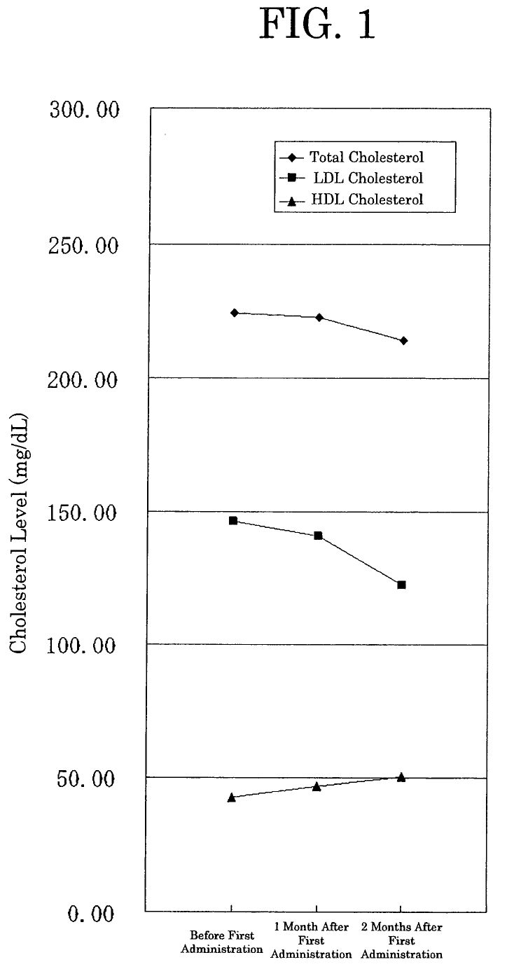

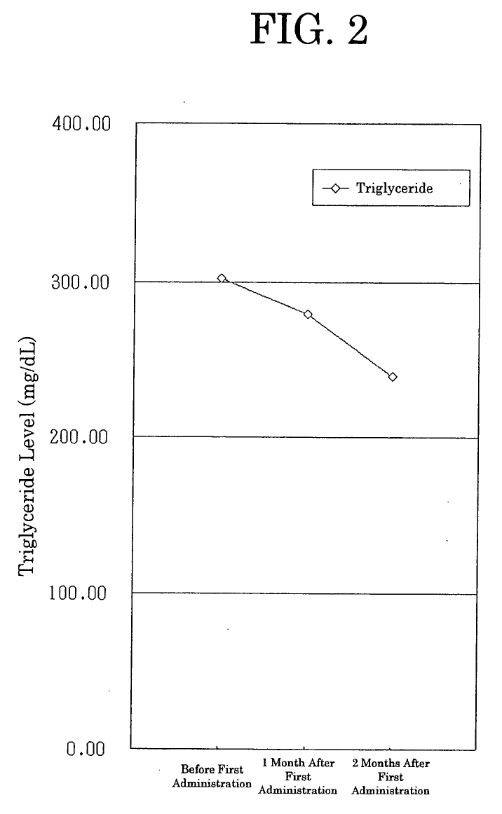

15 volunteers were dosed with the tablets produced in Example 1, with 6 tablets per day in three divided doses. And each LDL cholesterol level, HDL cholesterol level, total cholesterol level and triglyceride level before first administration, 1 month after the first administration and 2 months after the first administration were measured by LDL cholesterol direct method, HDL cholesterol direct method and enzymatic methods (CE-COD-POD method and GK-GPO method) respectively. The averages of each measurement of all volunteers were calculated from the obtained results. The average values are shown in Table 1. Moreover, changes in average values over time for LDL cholesterol level, HDL cholesterol level, total cholesterol level and triglyceride level are shown in FIGS. 1 and 2.

TABLE 1

| | Subject | Average |
|---|---|---|
| Example 1 | Age | 48.2 |
| LDL | Before Administration | 146.39 |
| cholesterol | After 1 Month | 140.74 |
| (mg/dl) | After 2 Months | 122.24 |
| HDL | Before Administration | 42.45 |
| cholesterol | After 1 Month | 46.74 |
| (mg/dl) | After 2 Months | 50.26 |
| Total | Before Administration | 224.44 |
| cholesterol | After 1 Month | 222.77 |
| (mg/dl) | After 2 Months | 214.09 |
| Triglyceride | Before Administration | 301.99 |
| (mg/dl) | After 1 Month | 279.07 |
| | After 2 Months | 239.11 |

From the results shown in Table 1, it turns out that HDL cholesterol level had been raised, and LDL cholesterol level had been lowered after 2 months compared to the first administration. Furthermore, because total cholesterol level had been lowered and triglyceride level, which is an indicator of neutral fat, had also been lowered, it turns out that the produced tablets have curative properties against hyperlipidemia.

The HDL cholesterol-raising agent of the present invention can raise or maintain HDL cholesterol level because it contains an olive extract as an active ingredient. Furthermore, the HDL cholesterol-raising agent can additionally have the LDL cholesterol-lowering effect. The HDL cholesterol-raising agent can be used in compositions, medicaments, foods, drinks and health foods, etc.

The LDL cholesterol-lowering agent of the present invention can lower LDL cholesterol level because it contains an olive extract as an active ingredient. The LDL cholesterol-lowering agent can be used in compositions, medicaments, foods, drinks and health foods, etc.

The composition of the present invention contains at least any one of HDL cholesterol-raising agent and LDL cholesterol-lowering agent. The compositions containing the HDL cholesterol-raising agent can selectively raise or maintain HDL cholesterol level. Moreover, the compositions containing the HDL cholesterol-raising agent can additionally have the LDL cholesterol-lowering effect. The compositions containing the LDL cholesterol-lowering agent have the LDL cholesterol-lowering effect. The compositions can be used as medicaments, foods, drinks and health foods.

The method for raising HDL cholesterol level in blood of the present invention can raise or maintain HDL cholesterol level in blood of mammals.

Moreover, the method for raising HDL cholesterol level and lowering LDL cholesterol level in blood of the present invention can raise or maintain HDL cholesterol level and lower LDL cholesterol level in blood of mammals.

By the present invention, it is possible to settle above existing issues and to provide [1] an HDL cholesterol-raising agent which can selectively raise or maintain HDL cholesterol level, [2] a LDL cholesterol-lowering agent which can selectively lower LDL cholesterol level, [3] a composition containing the HDL cholesterol-raising agent which can selectively raise or maintain HDL cholesterol level, [4] a method for raising HDL cholesterol level in blood which can selectively raise or maintain HDL cholesterol level in blood of mammals and [5] a method for raising HDL cholesterol level and lowering LDL cholesterol level in blood which selectively raises or maintains HDL cholesterol level and further selectively lowers LDL cholesterol level in blood of mammals.

1-10. (canceled)

11. A method for raising HDL cholesterol level in blood, comprising:
administration of an olive extract to mammals.

12. The method for raising HDL cholesterol level in blood according to claim 11, wherein the olive extract comprises Oleuropein.

13. A method for raising HDL cholesterol level and lowering LDL cholesterol level in blood, comprising:
administration of an olive extract to mammals.

14. The method for raising HDL cholesterol level and lowering LDL cholesterol level in blood according to claim 13, wherein the olive extract comprises Oleuropein.

15. A method for modifying the level of cholesterol in a mammal, said method comprising:
(a) administering an olive extract to the mammal; wherein the level of cholesterol in the mammal is modified in at least one manner selected from the group consisting of raising the HDL cholesterol level, lowering the LDL cholesterol level and combinations of the foregoing.

16. A method according to claim 15, wherein the olive extract comprises oleuropein.

17. A method according to claim 15, wherein the manner comprises lowering the LDL cholesterol level.

18. A method according to claim 15, wherein the manner comprises raising the HDL cholesterol level.

19. A method according to claim 16, wherein the manner comprises lowering the LDL cholesterol level.

20. A method according to claim 16, wherein the manner comprises raising the HDL cholesterol level.

21. A method for modifying the level of cholesterol in a mammal, said method comprising:
(a) administering an olive leaf extract to the mammal;
wherein the level of cholesterol in the mammal is modified in at least one manner selected from the group consisting of raising the HDL cholesterol level, lowering the LDL cholesterol level and combinations of the foregoing.

22. A method according to claim 21, wherein the olive leaf extract comprises oleuropein.

23. A method according to claim 21, wherein the manner comprises lowering the LDL cholesterol level.

24. A method according to claim 21, wherein the manner comprises raising the HDL cholesterol level.

25. A method according to claim 22, wherein the manner comprises lowering the LDL cholesterol level.

26. A method according to claim 22, wherein the manner comprises raising the HDL cholesterol level.
